(19) United States
(12) Patent Application Publication
(10) Pub. No.: US 2019/0078126 A1
(43) Pub. Date: Mar. 14, 2019

(54) POLYMERASE-MEDIATED, TEMPLATE-INDEPENDENT POLYNUCLEOTIDE SYNTHESIS

(71) Applicants: SIGMA-ALDRICH CO. LLC, St. Louis, MO (US); Merck Patent GmbH, Darmstadt (DE)

(72) Inventors: Thomas Baiga, Darmstadt (DE); Michael Anderson Burley, Darmstadt (DE); Alexander Smith, St. Louis, MO (US)

(21) Appl. No.: 16/125,448

(22) Filed: Sep. 7, 2018

Related U.S. Application Data
(60) Provisional application No. 62/556,083, filed on Sep. 8, 2017.
(60) Provisional application No. 62/556,090, filed on Sep. 8, 2017.

(51) Int. Cl. C12P 19/34; C12Q 1/6806; C12Q 1/6844

(52) U.S. Cl. CPC C12P 19/34; C07H 21/00; C12Q 1/6844; C12Q 1/6806

(57) ABSTRACT

Methods for de novo synthesis of polynucleotides in which 3′-O-reversibly blocked nucleotides are attached to a solid support in the presence of an X family DNA polymerase and in the absence of a nucleic acid template.

### CROSS REFERENCE TO RELATED APPLICATIONS

This application claims priority to U.S. Provisional Application Ser. No. 62/556,083, filed Sep. 8, 2017, and U.S. Provisional Application Ser. No. 62/556,090, filed Sep. 8, 2017, and the disclosure of each is hereby incorporated by reference in its entirety.

### FIELD

The present disclosure generally relates to methods for template independent de novo synthesis of polynucleotides.

### BACKGROUND

The synthesis and assembly of gene length DNA represents a significant bottleneck in modern biology. Oligonucleotide synthesis technologies are still based on chemistries developed in the 1970s and 1980s. In contrast, new and better DNA sequencing technologies have dramatically decreased the cost and increased the speed of sequencing. Thus, there is a need for new and improved polynucleotide synthesis methods that can quickly generate oligonucleotides or polynucleotides without the use of harsh chemical solvents.

### SUMMARY

Among the various aspects of the present disclosure are methods for template-independent, enzymatic synthesis of polynucleotides.

One aspect of the present disclosure is a method for synthesizing polynucleotides, wherein the method is template-independent and initiator sequence-independent. The method comprises (a) providing a solid support comprising a free hydroxyl group, wherein the free hydroxyl is part of a cleavable group linked to the solid support; (b) contacting the free hydroxyl group with a nucleotide 5′-triphosphate comprising a removable 3′-O-blocking group in the presence of an X family DNA polymerase and in the absence of a nucleic acid template to form an immobilized nucleotide comprising a removable 3′-O-blocking group; (c) contacting the immobilized nucleotide comprising the removable 3′-O-blocking group with a deblocking agent to remove the removable 3′-O-blocking group; (d) repeating steps (b) and (c) to yield the polynucleotide; and (e) cleaving the cleavable group of the solid support to release the polynucleotide.

Another aspect of the present disclosure encompasses a template-independent method for synthesizing polynucleotides. The method comprises (a) providing a nucleotide comprising a free 3′-OH group; (b) contacting the free 3′-OH group with a nucleotide 5′-triphosphate comprising a removable 3′-O-blocking group in the presence of an X family DNA polymerase and in the absence of a nucleic acid template to form an oligonucleotide comprising a removable 3′-O-blocking group, wherein the removable 3′-O-blocking group of the nucleotide 5′-triphosphate is chosen from (CO)R, (CO)OR, or (CO)CH₂OR, wherein R is alkyl or alkenyl, provided that the removable 3′-O-blocking group is other than acetyl; (c) contacting the oligonucleotide comprising the removable 3′-O-blocking group with a deblocking agent to remove the removable 3′-O-blocking group; and (d) repeating steps (b) and (c) to yield the polynucleotide.

Other aspects and iterations of the disclosure are detailed below.

### BRIEF DESCRIPTION OF THE DRAWINGS

FIG. 1 presents a schematic diagram of a polymerase-mediated, template-independent, initiator sequence-independent polynucleotide synthesis method disclosed herein. As detailed below, L is a linker, PC is a cleavable group, W is blocking group, and B is a base or analog thereof.

FIG. 2 presents a schematic diagram of a polymerase-mediated, template-independent polynucleotide synthesis method.

FIG. 3A illustrates template-independent incorporation of 3′-O-carbamate or ester blocked nucleotides (dNTP-1, -2, -3, -5, -6) into a primer in solution by Bt TdT.

FIG. 3B shows template-independent incorporation of 3′-O-carbamate or ester blocked nucleotides (dNTP-1, -2, -3, -5, -6) into a primer on a solid support by Bt TdT.

FIG. 4 presents template-independent incorporation of 3′-O-carbamate or ester blocked nucleotides by a modified X family DNA polymerase, i.e., a PolM-loop 1 chimera.

FIG. 5 shows multiple cycles of incorporation (and deblocking) by the PolM-loop 1 chimera.

### DETAILED DESCRIPTION

The present disclosure provides polymerase-mediated, template-independent methods for synthesizing polynucleotides. The methods utilize a step of linking 3′-O-reversibly blocked nucleotides 5′-triphosphates to a free hydroxyl group in the presence of an X family DNA polymerase and absence of a nucleic acid template, followed by a step of deblocking or removing the 3′-O-blocking group to create a free hydroxyl group. The method comprises repeating the steps of linking and deblocking to form the polynucleotide of the desired sequence. Advantageously, the steps of the polynucleotide synthesis method are conducted in the presence of aqueous solutions, thereby providing a green chemistry method.

### (I) Methods for Template-Independent, Initiator Sequence-Independent Polynucleotide Synthesis

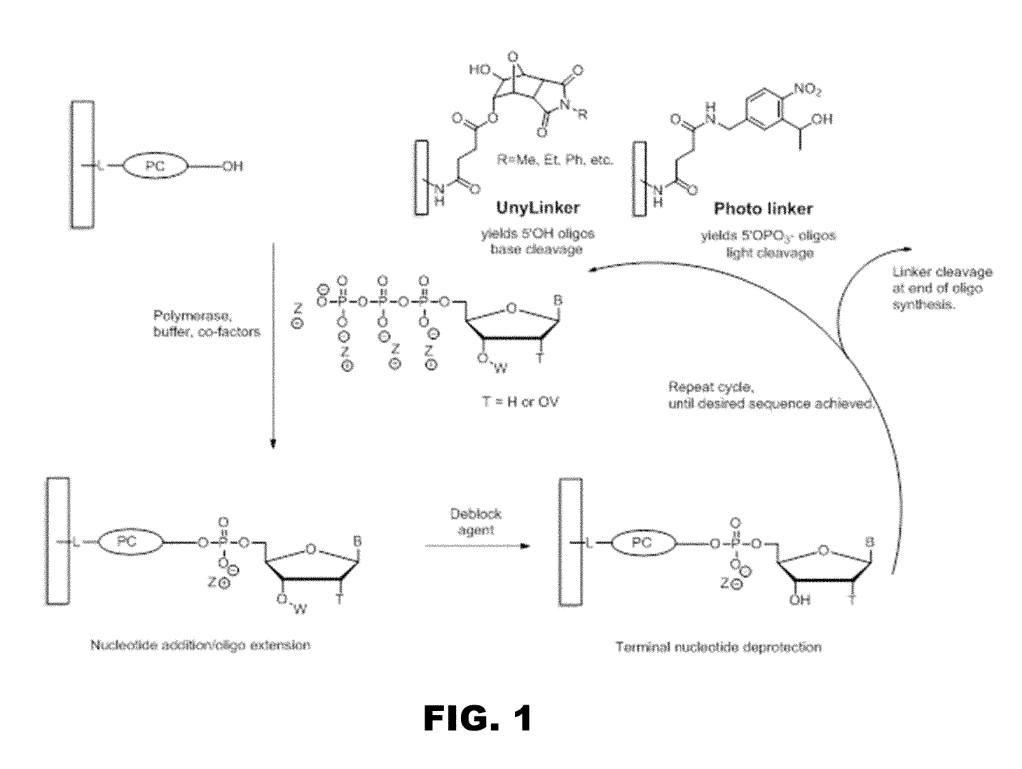

One aspect of the present disclosure provides template-independent and initiator sequence-independent methods for de novo synthesis of polynucleotides. In particular, the methods comprise (a) providing a solid support comprising a covalently attached cleavable linker comprising a free hydroxyl group; (b) contacting the free hydroxyl group with a nucleotide 5′-triphosphate comprising a removable 3′-O-blocking group in the presence of an X family DNA polymerase and absence of a nucleic acid template to form an immobilized nucleotide comprising a removable 3′-O-blocking group; (c) contacting the immobilized nucleotide comprising a removable 3′-O-blocking group with a deblocking agent to remove the removable 3′-O-blocking group; (d) repeating steps (b) and (c) to yield the polynucleotide of the desired sequence; and (e) cleaving the cleavable linker of the solid support to release the polynucleotide. FIG. 1 presents a reaction scheme depicting this polynucleotide synthesis process.

(a) Reactants

The template-independent, initiator sequence-independent polynucleotide synthesis methods commence with formation of a reaction phase comprising a solid support comprising a free hydroxyl group, a nucleotide 5′-triphosphase comprising a removable 3′-O-blocking group, and an X family DNA polymerase, each of which is detailed below. This method allows for polymerase-mediated synthesis of polynucleotides without the use of a nucleic acid template and without the use of a primer or initiator sequence.

(i) Solid Support Comprising Free Hydroxyl Group

In general, the solid support comprises a free hydroxyl group, such that the oxygen of the free hydroxyl group can be linked via a phosphodiester bond to the alpha phosphate of a nucleotide 5′-triphosphate comprising a removable 3′-O-blocking group. In some embodiments, the free hydroxyl group is part of a cleavable group (PC) that is attached to the solid support via a linker (L), as diagrammed below:

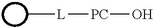

A variety of cleavable groups are suitable for linking to the solid support. The cleavable group can be cleaved by any of several mechanisms. For example, the cleavage group can be acid cleavable, base cleavable, photocleavable, electophilically cleavable, nucleophilically cleavable, cleavable under reduction conditions, cleavable under oxidative conditions, or cleavable by elimination mechanisms. Those skilled in the art are familiar with suitable cleavage sites, such as, e.g., ester linkages, amide linkages, silicon-oxygen bonds, trityl groups, tert-butyloxycarbonyl groups, acetal groups, p-alkoxybenzyl ester groups, and the like.

In specific embodiments, the cleavable group can be a photocleavable group, wherein cleavage is activated by light of a particular wavelength. Non-limiting examples of suitable photocleavable groups include nitrobenzyl, nitrophenethyl, benzoin, nitroveratryl, phenacyl, pivaloyl, sisyl, 2-hydroxy-cinamyl, coumarin-4-yl-methyl groups or derivatives thereof. In particular embodiments, the photocleavable group can be a member of the ortho-nitrobenzyl alcohol family and attached to linker L as diagrammed below.

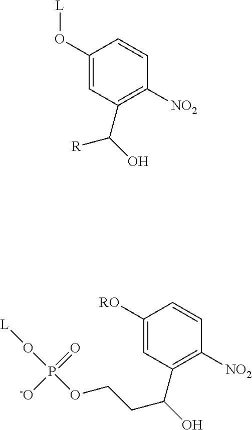

In other embodiments, the cleavable group can be a base hydrolysable group attached to linker L, as diagrammed below, wherein R can be alkyl, aryl, etc.

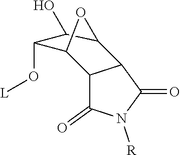

The linker (L) can be any bifunctional molecule comprising from about 6 to about 100 contiguous covalent bond lengths. For example, the linker can be an amino acid, a peptide, a nucleotide, a polynucleotide (e.g., poly A₃₋₂₀), an abasic sugar-phosphate backbone, a polymer (e.g., PEG, PLA, cellulose, and the like), a hydrocarbyl group (e.g., alkyl, alkenyl, alkynyl, aryl, aralkyl, aralkenyl, aralkynyl, and so forth), a substituted hydrocarbyl group (e.g., alkoxy, heteroaryl, aryloxy, and the like), or a combination thereof.

Specific solid supports in which the free hydroxyl group is part of a photocleavable group that is attached to the solid support via a linker (L) are diagrammed below.

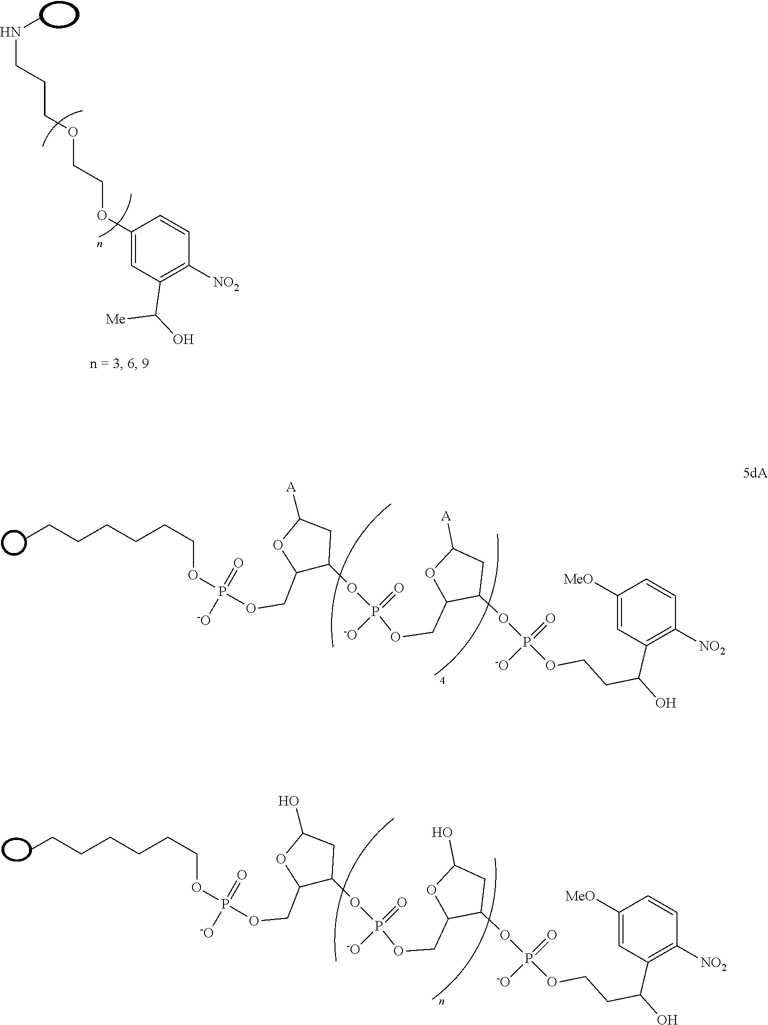

In various embodiments, the solid support can be a bead, a well, a plate, a chip, a microplate, an assay plate, a testing plate, a slide, a microtube, or any other suitable surface. The solid support can comprise polymer, plastic, resin, silica, glass, silicon, metal, carbon, or other suitable material. In certain embodiments, the solid support can be a polymer. Non-limiting examples of suitable polymers include polypropylene, polyethylene, cyclo-olefin polymer (COP), cyclo-olefin copolymer (COC), polystyrene, and polystyrene crosslinked with divinylbenzene. In specific embodiments, the polymer can be polypropylene, cyclo-olefin polymer, or cyclo-olefin copolymer.

(ii) 3′-O-Reversibly Blocked Nucleotide 5′-Triphosphates

The reaction phase also comprises a nucleotide 5′-triphosphate comprising a removable 3′-O-blocking group. A nucleotide comprises a nitrogenous base, a sugar moiety (i.e., ribose, 2′-deoxyribose, or 2′-4′ locked deoxyribose), and one or more phosphate groups. The removable 3′-O-blocking group can be an ester, ether, carbonitrile, phosphate, carbonate, carbamate, hydroxylamine, borate, nitrate, sugar, phosphoramide, phosphoramidate, phenylsulfonate, sulfate, sulfone, or amino acid.

The nucleotide 5′-triphosphate comprising the removable 3′-O-blocking group can be a deoxyribonucleotide, a ribonucleotide, or a locked nucleic acid (LNA), respectively, as diagrammed below:

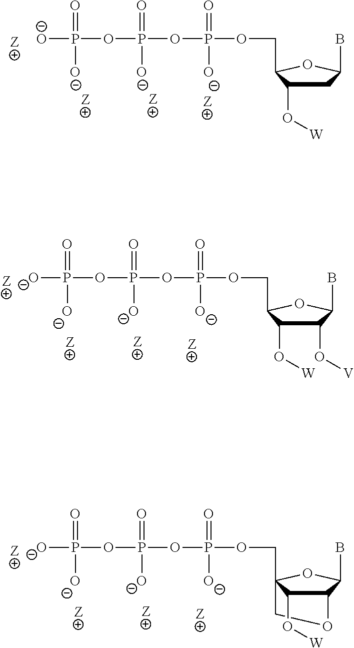

wherein:
B is a nitrogenous base;
W is a removable blocking group chosen from (CO)R, (CO)OR, (CO)CH₂OR, (CO)NHR, (CO)CH₂NHR, (CO)SR, CH₂OR, CH₂N₃, CH₂CH═CH₂, CH₂CN, NH₂, NH₃⁺X⁻, NR₃⁺X⁻, NHR, NRR¹, NO₂, BO₃, SOR, SO₂R, SO₃R, PO₃X₃, SiRR¹R², 2-furanyl, 2-thiofuranyl, 3-pyranyl, or 2-thiopyranylo, wherein R, R¹, and R² independently are alkyl, alkenyl, aryl, substituted alkyl, substituted alkenyl, or substituted aryl, and X is an anion;
V is hydrogen, SiRR¹R², or CH₂OSiRR¹R², wherein R, R¹, and R² independently are alkyl, alkenyl, aryl, substituted alkyl, substituted alkenyl, or substituted aryl; and
Z is a cation.

In various embodiments, B can be a standard nucleobase, a non-standard base, a modified base, an artificial (or unnatural) base, or analog thereof. Standard nucleobases include adenine, guanine, thymine, uracil, and cytosine. In other embodiments, B can be 2-methoxy-3-methylnapthlene (NaM), 2,6-dimethyl-2H-isoquinoline-1-thione (5SICS), 8-oxo guanine (8-oxoG), 8-oxo adenine (8-oxoA), 5-methylcytosine (5mC), 5-hydroxymethyl cytosine (5hmC), 5-formyl cytosine (5fC), 5-carboxy cytosine (5caC), xanthine, hypoxanthine, 2-aminoadenine, 6-methyl or 6-alkyl adenine, 6-methyl or 6-alkyl guanine, 2-propyl or 2-alkyl adenine, 2-propyl or 2-alkyl guanine, 2-thiouracil, 2-thiothymine, 2-thiocytosine, 5-propynyl uracil, 5-propynyl cytosine, 6-azo uracil, 6-azo cytosine, 6-azo thymine, 5-uracil (pseudouracil), 4-thiouracil, 8-halo (e.g., 8-bromo) adenine, 8-amino adenine, 8-thiol adenine, 8-thioalkyl adenine, 8-hydroxyl adenine, 8-halo (e.g., 8-bromo) guanine, 8-amino guanine, 8-thiol guanine, 8-thioalkyl guanine, 8-hydroxyl guanine, 5-halo (e.g., 5-bromo) uracil, 5-trifluoromethyl uracil, 5-halo (e.g., 5-bromo) cytosine, 5-trifluoromethyl cytosine, 7-methylguanine, 7-methyladenine, 8-azaguanine, 8-azaadenine, deazaguanine, 7-deazaguanine, 3-deazaguanine, deazaadenine, 7-deazaadenine, 3-deazaadenine, pyrazolo[3,4-d]pyrimidine, inosine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine, 1,3,5 triazine, FEMO, MMO2, or TPT3.

In general, Z can be an alkali metal, an alkaline earth metal, a transition metal, NH₄, or NR₄, wherein R is alkyl, aryl, substituted alkyl, or substituted aryl. Suitable metals include sodium, potassium, lithium, cesium, magnesium, calcium, manganese, cobalt, copper, zinc, iron, and silver. In specific embodiments, Z can be lithium or sodium.

In certain embodiments, W can be (CO)R, (CO)OR, or (CO)CH₂OR, wherein R is alkyl or alkenyl. For example, W can be (CO)—O-methyl, (CO)—O-ethyl, (CO)—O-n-propyl, (CO)—O-isopropyl, (CO)—O-propenyl, (CO)—O-n-butyl, (CO)—O-t-butyl, (CO)CH₂O-methyl, (CO)CH₂O-ethyl, (CO)CH₂O-n-propyl, (CO)CH₂O-isopropyl, (CO) CH₂O-n-butyl, (CO) CH₂O-t-butyl, (CO)methyl, (CO)ethyl, (CO)n-propyl, (CO)isopropyl, (CO)n-butyl, or (CO)t-butyl. In specific embodiments, W can be (CO)—O-methyl, (CO)—O-ethyl, (CO)ethyl, (CO)n-propyl, (CO)CH₂O-methyl, or (CO)CH₂O-ethyl.

In certain embodiments, the 3′-O-reversibly blocked nucleotide 5′-triphosphate can further comprise a detectable label. The detectable label can be a detection tag such as biotin, digoxigenin, or dinitrophenyl, or a fluorescent dye such as fluorescein or derivatives thereof (e.g., FAM, HEX, TET, TRITC), rhodamine or derivatives thereof (e.g., ROX), Texas Red, cyanine dyes (e.g., Cy2, Cy3, Cy5), Alexa dyes, diethylaminocoumarin, and the like. In some embodiments, the detectable label can comprise a fluorescent dye-quencher pair. Non-limiting examples of suitable quenchers include black hole quenchers (e.g., BHQ-1, BHQ-3), Iowa quenchers, deep dark quenchers, eclipse quenchers, and dabcyl. The detectable label can be attached directly to the nitrogenous base or can be attached via a chemical linker. Suitable chemical linkers include tetra-ethylene glycol (TEG) spacers, polyethylene glycol (PEG) spacers, C6 linkers, and other linkers known in the art.

(iii) X Family of DNA Polymerases

The reaction phase also comprises an X family DNA polymerase, wherein the X family DNA polymerase can accommodate 3′-O-blocked nucleotide 5′-triphosphates and is capable of incorporating 3′-O-blocked nucleotides in the absence of a nucleic acid template.

Suitable X family DNA polymerase members include terminal deoxynucleotidyl transferase (TdT), DNA polymerase beta (DNA pol β), DNA polymerase lambda (DNA pol λ), DNA polymerase mu (DNA pol μ), DNA polymerase theta (DNA pol θ), and DNA polymerase X. The X family DNA polymerase can be of eukaryotic, viral, archaeal, or bacterial origin. The X family DNA polymerase can be wild type, a truncated version, or a modified (i.e., engineered) version thereof.

In some embodiments, the X family DNA polymerase can be human TdT, bovine TdT, primate TdT, porcine TdT, mouse TdT, marsupial TdT, rodent TdT, canine TdT, chicken TdT, truncated versions of any of the foregoing, or modified versions of any of the foregoing. In other embodiments, the X family DNA polymerase can be a modified DNA polymerase beta, a modified DNA polymerase lambda, a modified DNA polymerase mu, a modified DNA polymerase theta, or a modified DNA polymerase X that has been engineered to be capable of template independent nucleic acid synthesis. The modified DNA polymerase beta, DNA polymerase lambda, DNA polymerase mu, or DNA polymerase theta can be of mammalian origin (e.g., human, primate, mouse, etc.), as well as vertebrate (e.g., fish, frog, etc.), invertebrate, fungal, or plant origin. The modified DNA polymerase X can be from African swine fever virus (ASFV).

In certain embodiments, the X family DNA polymerase can be derived from human DNA polymerase beta (UniprotKB No. P06746, DPOLB_Human) or an ortholog thereof. In other embodiments, the X family DNA polymerase can be derived from human DNA polymerase lambda (UniprotKB No. Q9UGP5, DPOLL_Human) or an ortholog thereof. In still other embodiments, the X family DNA polymerase can be derived from human DNA polymerase mu (UniprotKB No. Q9NP87, DPOLM_Human) or an ortholog thereof. In other embodiments, the X family DNA polymerase can be derived from human DNA polymerase theta (UniprotKB No. 075417, DPOLQ_Human) or an ortholog thereof. In yet other embodiments, the X family DNA polymerase can be derived from African swine fever virus (ASFV) DNA polymerase X (UniprotKB No. P42494, DPOLX_ASFB7) or an ortholog thereof.

In various embodiments, the X family DNA polymerase can be modified to have increased activity in the presence of nucleotide triphosphates bearing 3′-O-blocking groups (i.e., increased incorporation of the 3′-O-blocked nucleotides) or increased activity in the absence of a template. The modification can comprise one or more mutations in one or more regions of the X family DNA polymerase including, but not limited to, the active sites, the secondary shell, the surface, the Loop 1 motif, and the non-loop 1 primary shelf. The mutations can be substitutions of one or more amino acids (e.g., substitution of alanine for another amino acid), insertions of one or more amino acids, and/or deletions of one or more amino acids within the protein and/or at one or both ends of the X family DNA polymerase. In particular embodiments, the modified X family DNA polymerase can comprise an insertion/swap of a TdT Loop 1 motif into the corresponding region. In additional embodiments, the modified X family DNA polymerase can comprise the Loop 1 insertion in combination with an N-terminal truncation.

In some embodiments, the modified X family DNA polymerase can further comprise at least one marker domain and/or purification tag. Non-limiting examples of marker domains include fluorescent proteins, purification tags, and epitope tags. In some embodiments, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalama1, GFPuv, Sapphire, T-sapphire), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. Examples of purification tags include, without limit, poly-His, FLAG, HA, tandem affinity purification (TAP), glutathione-S-transferase (GST), chitin binding protein (CBP), maltose binding protein, thioredoxin (TRX), poly(NANP), myc, AcV5, AU1, AU5, E, ECS, E2, nus, Softag 1, Softag 3, Strep, SBP, Glu-Glu, HSV, KT3, S, S1, T7, V5, VSV-G, biotin carboxyl carrier protein (BCCP), and calmodulin. The marker domain and/or purification can be located at the N-terminal end and/or the C-terminal end of the modified polymerase.

(b) Steps of the Process

The template-independent polynucleotide synthesis method comprises cycles of linking a 3′-O-reversibly blocked nucleotide and removing the reversible 3′-O-blocking group so that another 3′-O-reversibly blocked nucleotide can be linked to the elongating polynucleotide.

(i) Linking 3′-O-Reversibly Blocked Nucleotides

The template-independent, initiator sequence-independent polynucleotide synthesis methods disclosed herein comprise a linking step in which a nucleotide comprising a removable 3′O-blocking group is linked to a solid support comprising a free hydroxyl group. The linking step comprises reacting the free hydroxyl group with a nucleotide 5′-triphosphate comprising a removable 3′-O-blocking group in the presence of an X family DNA polymerase and in the absence of a nucleic acid template. The X family DNA polymerase links the alpha 5′-phosphate group of the 3′-O-blocked nucleotide to the oxygen of the free hydroxyl group of the solid support via a phosphodiester bond. The 3′-O-blocking group of the newly linked nucleotide prevents the addition of additional nucleotides to the oligo/polynucleotide.

The linking step generally is conducted in the presence of an aqueous solution. The aqueous solution can comprise one or more buffers (e.g., Tris, HEPES, MOPS, Tricine, cacodylate, barbital, citrate, glycine, phosphate, acetate, and the like) and one or more monovalent and/or divalent cations (e.g., Mg²⁺, Mn²⁺, Co²⁺, Cu²⁺, Zn²⁺, Na⁺, K⁺, etc. along with an appropriate counterion, such as, e.g., Cl⁻). In some embodiments, the aqueous solution can further comprise one or more nonionic detergents (e.g., Triton X-100, Tween-20, and so forth). In other embodiments, the aqueous solution can further comprise an inorganic pyrophosphatase (to counter the levels of pyrophosphate due to nucleotide triphosphate hydrolysis). The inorganic pyrophosphatase can be of yeast or bacterial (e.g., E. coli) origin. The aqueous solution generally has a pH raging from about 5 to about 10. In certain embodiments, the pH of the aqueous solution can range from about 6 to about 9, from about 6 to about 7, from about 7 to about 8, or from about 7 to about 9.

The linking step can be conducted at a temperature ranging from about 4° C. to about 80° C. In various embodiments, the temperature can range from about 4° C. to about 20° C., from about 20° C. to about 40° C., from about 40° C. to about 60° C., or from about 60° C. to about 80° C. In specific embodiments, the temperature of the linking step can range from about 20° C. to about 50° C., or from about 25° C. to about 40° C.

During the linking step, the nucleotide 5′-triphosphate comprising the removable 3′-O-blocking group can be present at a concentration ranging from about 1 μM to about 1 M. In certain embodiments, the concentration of the nucleotide 5′-triphosphate comprising a removable 3′-O-blocking group can range from about 1 μM to about to about 10 μM, from about 10 μM to about 100 μM, or from about 100 μM to about 1000 μM. The weight ratio of the solid support comprising the free hydroxyl group to the nucleotide 5′-triphosphate comprising the removable 3′-O-blocking group can range from about 1:100 to about 1:10,000. In specific embodiments, the weight ratio of the solid support comprising the free hydroxyl group to the nucleotide 5′-triphosphate comprising the removable 3′-O-blocking group can range from about 1:500 to about 1:2000.

In general, the amount of the X family DNA polymerase present during the linking step will be sufficient to catalyze the reaction in a reasonable period of time. In general, the linking step is allowed to proceed until the phosphodiester bond formation is complete. The formation of the phosphodiester bond can be monitored by incorporating a 3′-O-blocked nucleotide comprising a fluorescent label.

At the end of the linking step, the X family DNA polymerase and the unreacted 3′-O-reversibly blocked nucleotide 5′-triphosphate generally are removed from the immobilized nucleotide. In some embodiments, the aqueous solution comprising the X family DNA polymerase and the unreacted 3′-O-reversibly blocked nucleotide 5′-triphosphate can be removed, optionally recycled, and replaced with aqueous solution (e.g., fresh or recycled aqueous solution that is used during the deblocking step, described below). In other embodiments, the X family DNA polymerase can be removed from the aqueous solution by contact with an antibody that recognizes the X family DNA polymerase. In still other embodiments, the aqueous solution comprising the X family DNA polymerase and/or the unreacted 3′-O-reversibly blocked nucleotide 5′-triphosphate can be washed or flushed away with a wash solution. The wash solution can comprise the same components as used during the deblocking step.

(ii) Removing the 3′-O-Removable Blocking Group

The method further comprises a deblocking step in which the removable 3′-O-blocking group is removed from the 3′-O-blocked nucleotide immobilized on the solid support. The deblocking step comprises contacting the immobilized nucleotide comprising the removable 3′-O-blocking group with a deblocking agent, thereby removing the 3′-O-blocking group and creating a free hydroxyl group on the immobilized nucleotide (or polynucleotide).

The type and amount of deblocking agent will depend upon the identity of the removable 3′-O-blocking group. Suitable deblocking agents include acids, bases, nucleophiles, electrophiles, radicals, metals, reducing agents, oxidizing agents, enzymes, and light. In embodiments in which the blocking group comprises an ester or carbamate linkage, the deblocking agent can be a base (e.g., an alkali metal hydroxide). In instances in which the blocking group comprises an ether linkage, the deblocking agent can be an acid. In embodiments in which when the blocking group is O-amino, the deblocking agent can be sodium nitrite. In aspects in which the blocking group is O-allyl, the deblocking agent can be a transition metal catalyst. In embodiments in which the blocking group is azidomethyl, the deblocking agent can be a phosphine (e.g., tris(2-carboxyethyl)phosphine). In embodiments in which the blocking group comprises an ester or carbonate linkage, the deblocking agent can be an esterase or lipase enzyme. The esterase or lipase enzyme can be derived from animal, plant, fungi, archaeal, or bacterial sources. The esterase or lipase can be mesophilic or thermophilic. In one embodiment, the esterase can be derived from porcine liver.

In general, the deblocking step is conducted in the presence of an aqueous solution. That is, the deblocking agent can be provided as an aqueous solution comprising the deblocking agent. In some embodiments, the aqueous solution can comprise one or more protic, polar solvents. Suitable protic, polar solvents include water; alcohols such as methanol, ethanol, isopropanol, n-propanol, isobutanol, n-butanol, s-butanol, t-butanol, and the like; diols such as glycerol, propylene glycol and so forth; organic acids such as formic acid, acetic acid, and so forth; an amine such as triethylamine, morpholine, piperidine, and the like; and combinations of any of the above. In other embodiments, the aqueous solution can comprise one or more buffers (e.g., Tris, HEPES, MOPS, Tricine, cacodylate, barbital, citrate, glycine, phosphate, acetate, and the like). In still other embodiments, the aqueous solution can further comprise one or more denaturants to disrupt any secondary structures in the oligo/polynucleotides. Suitable denaturants include urea, guanidinium chloride, formamide, and betaine.

The pH of the aqueous solution can range from about 1 to about 14, depending upon the identity of the deblocking agent. In various embodiments, the pH of the aqueous solution can range from about 2 to about 13, from about 3 to about 12, from about 4 to about 11, from 5 to about 10, from about 6 to about 9, or from about 7 to about 8. In specific embodiments, the pH of the aqueous solution comprising the deblocking agent can range from about 10 to about 14, or from about 11 to about 13.

In embodiments in which the deblocking agent is an esterase or lipase enzyme, the enzyme can be provided in a buffered aqueous solution having a pH from about 6.5 to about 8.5.

The deblocking step can be performed at a temperature ranging from about 0° C. to about 100° C. In some embodiments, the temperature can range from about 4° C. to about 90° C. In various embodiments, the temperature can range from about 0° C. to about 20° C., from about 20° C. to about 40° C., from about 40° C. to about 60° C., from about 60° C. to about 80° C., or from about 80° C. to about 100° C. In certain embodiments, then deblocking step can be performed at about 60° C. to about 80° C. The deblocking step can be performed at a first temperature, followed by a second temperature. For example, the aqueous solution comprising the deblocking agent can be provided at one temperature and then the temperature can be raised to assist in cleavage and disrupt any secondary structure.

The duration of the deblocking step will vary depending upon the nature of the protecting chemistry and type of deblocking agent. In general, the deblocking step is allowed to proceed until the reaction has gone to completion, as determined by methods known in the art.

At the end of the deblocking step, the deblocking agent generally is removed from the immobilized nucleotide bearing the free hydroxyl group. In some embodiments, the aqueous solution comprising the deblocking agent can be removed, optionally recycled, and replaced with aqueous solution (e.g., fresh or recycled aqueous solution that is used during the linking step, as described above). In other embodiments, the aqueous solution comprising the deblocking agent can be washed or flushed away with a wash solution. The wash solution can comprise the same buffers and salts as used during the linking step. In embodiments in which the deblocking agent is an enzyme, the enzyme can be removed from the aqueous solution by contact with an antibody that recognizes the enzyme.

In specific embodiments, the removable 3′-O-blocking group is linked to the nucleotide 5′-triphosphase via an ester or carbonate linkage, and the deblocking agent is a base or an esterase or lipase enzyme.

(iii) Repeating the Linking and Deblocking Steps

The steps of linking 3′-O-blocked nucleotides to the immobilized nucleotide (or polynucleotide) and removing the removable blocking group can be repeated until the polynucleotide of the desired length and sequence is achieved.

The linking and deblocking steps can be performed in a microfluidic instrument, a column-based flow instrument, or an acoustic droplet ejection (ADE)-based system. The aqueous solution comprising the appropriate 3′-O-blocked nucleotide 5′-triphosphate and the X family DNA polymerase, the aqueous solution comprising the deblocking agent, wash solutions, etc., can be dispensed through acoustic transducers or microdispensing nozzles using any applicable jetting technology, including piezo or thermal jets. The temperature and duration of each step can be controlled by a processing unit.

(iv) Releasing the Polynucleotide

The final step of the polynucleotide synthesis methods disclosed herein comprises cleaving the cleavable group linked to the solid support to release the polynucleotide.

Cleavable groups and means for cleaving said groups are detailed above in section (I)(a)(i). In certain embodiments, the cleavage group can be cleaved by contact with a base (i.e., an alkaline solution). In specific embodiments, the cleavable group is a photocleavable group that can be cleaved by contact with light of a suitable wavelength. The released polynucleotide can have a 5′-hydroxyl group or a 5′-phosphoryl group.

The polynucleotides synthesized by the methods described herein can be deoxyribonucleic acid (DNA), ribonucleic acid (RNA), locked nucleic acid (LNA), or a combination thereof. In general, the polynucleotides prepared by the methods disclosed herein are single stranded. In embodiments in which the polynucleotide is DNA, the single-stranded DNA can be converted to double-stranded DNA by contact with a DNA polymerase (as well as suitable primers and dNTPs). The DNA polymerase can be thermophilic or mesophilic. Suitable DNA polymerases include Taq DNA polymerase, Pfu DNA polymerase, Pfx DNA polymerase, Tli (also known as Vent) DNA polymerase, Tfl DNA polymerase, Tth DNA polymerase, Tko DNA polymerase (also known as KOD), E. coli DNA polymerase I, T4 DNA polymerase, T7 DNA polymerase, variants thereof, and engineered versions thereof.

The lengths of polynucleotides synthesized by the methods described herein can range from about several nucleotides (nt) to hundreds of thousands or millions of nt. In various embodiments, the polynucleotide can comprise from about 4 nt to about 30 nt, from about 30 nt to about 100 nt, from about 100 nt to about 300 nt, from about 300 nt to about 1000 nt, from about 1000 nt to about 3000 nt, from about 3,000 nt to about 10,000, from about 10,000 nt to about 100,000 nt, from about 100,000 nt to about 1,000,000 nt, or from about 1,000,000 nt to about 10,000,000 nt.

As such, the methods disclosed herein can be used to synthesize whole genes or synthetic genes for research, clinical, diagnostic, and/or therapeutic applications. Similar, the methods disclosed herein can be used to synthesize whole plasm ids, synthetic plasm ids, and/or synthetic viruses (e.g., DNA or RNA) for a variety of applications. Additionally, the methods disclosed herein can be used to synthesize long synthetic RNAs for a variety of research and/or diagnostic/therapeutic applications.

### (II) Methods for Template-Independent Polynucleotide Synthesis

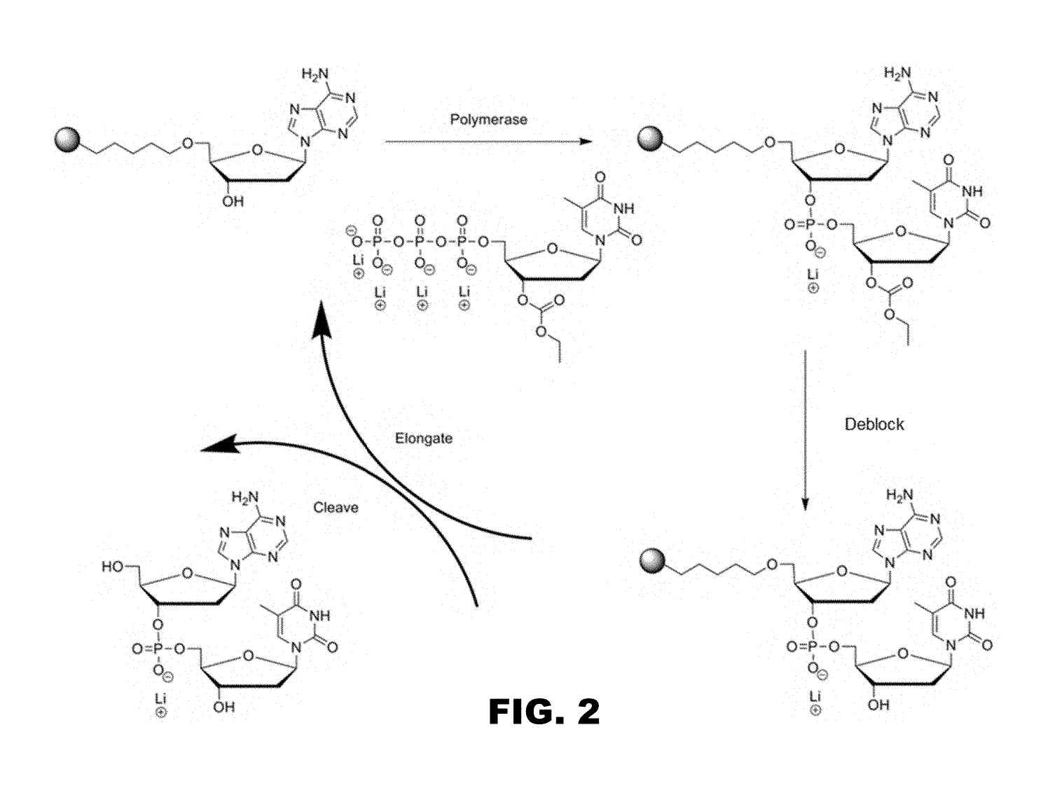

Another aspect of the present disclosure encompasses additional template-independent methods for synthesis of polynucleotides. Such methods comprise (a) providing a nucleotide comprising a free 3′-OH group; (b) contacting the free 3′-OH group with a nucleotide 5′-triphosphate comprising a removable 3′-O-blocking group in the presence of an X family DNA polymerase and absence of a nucleic acid template to form an immobilized oligonucleotide comprising a removable 3′-O-blocking group; (c) contacting the immobilized oligonucleotide comprising a removable 3′-O-blocking group with a deblocking agent to remove the removable 3′-O-blocking group; and (d) repeating steps (b) and (c) to yield the polynucleotide of the desired sequence. FIG. 2 presents a reaction scheme showing this polynucleotide synthesis process.

(a) Reactants

This polynucleotide synthesis method commences with formation of a reaction phase comprising a nucleotide comprising a free 3′-OH group, a nucleotide 5′-triphosphase comprising a 3′-O-blocking group, and an X family DNA polymerase that is other than a terminal deoxynucleotidyl transferase or a modified version thereof.

(i) Nucleotide Comprising a Free 3′-OH Group

The nucleotide comprising a free 3′-OH group provides the site for attachment of the incoming nucleotide via formation of a phosphodiester bond with the alpha phosphate of the nucleotide 5′-triphosphate comprising the 3′-O-blocking group. In some embodiments, the nucleotide comprising the free 3′-OH group can be located at the 3′ end of primer or initiator sequence. The primer or initiator sequence can be immobilized on a solid support. In other embodiments, the nucleotide comprising the free 3′-OH group can be located at the 3′ end of an elongating polynucleotide. The elongating polynucleotide can be immobilized on a solid support.

(ii) 3′-O-Reversibly Blocked Nucleotide 5′-Triphosphates

The reaction phase also comprises a nucleotide 5′-triphosphase comprising a removable 3′-O-blocking group. Examples of 3′-O-reversibly blocked nucleotide 5′-triphosphates are detailed above in section (I)(a)(ii). In general, the 3′-O-blocking group is chosen from (CO)R, (CO)OR, or (CO)CH₂OR, wherein R is alkyl or alkenyl, provided that the 3′-O-blocking group is other than acetyl. In various embodiments, the 3′-O-blocking group can be (CO)—O-methyl, (CO)—O-ethyl, (CO)—O-n-propyl, (CO)—O-isopropyl, (CO)—O-propenyl, (CO)—O-n-butyl, (CO)—O-t-butyl, (CO)CH₂O-methyl, (CO)CH₂O-ethyl, (CO)CH₂O-n-propyl, (CO)CH₂O-isopropyl, (CO) CH₂O-n-butyl, (CO) CH₂O-t-butyl, (CO)ethyl, (CO)n-propyl, (CO)isopropyl, (CO)n-butyl, or (CO)t-butyl. In specific embodiments, the 3′-O-blocking group can be (CO)—O-methyl, (CO)—O-ethyl, (CO)ethyl, (CO)propyl, (CO)CH₂O-methyl, or (CO)CH₂O-ethyl.

The sugar moiety of the 3′-O-reversibly blocked nucleotide 5′-triphosphate can be ribose, 2′-deoxyribose, or 2′-4′ locked deoxyribose, and the nitrogenous base of the nucleotide can be a standard nucleobase, a non-standard base, a modified base, an artificial (or unnatural) base, or analog thereof, examples of which are described above in section (I)(a)(ii).

(iii) X family DNA Polymerase

The reaction phase further comprises an X family DNA polymerase, examples of which are detailed above in section (I)(a)(iii).

(b) Steps of the Process

The synthesis method comprises linking and deblocking steps as described above in sections (I)(b)(i)-(iii). In embodiments in which the newly synthesized polynucleotide is attached to a solid support, the method can further comprise releasing the polynucleotide from the solid support using methods known in the art.

### ENUMERATED EMBODIMENTS

The following enumerated embodiments are presented to illustrate certain aspects of the present invention, and are not intended to limit its scope.

1. A method for synthesizing a polynucleotide, wherein the method is template-independent and initiator sequence-independent, and the method comprises (s) providing a solid support comprising a free hydroxyl group, wherein the free hydroxyl group is part of a cleavable group linked to the solid support; (b) contacting the free hydroxyl group with a nucleotide 5′-triphosphate comprising a removable 3′-O-blocking group in the presence of an X family DNA polymerase and absence of a nucleic acid template to form an immobilized nucleotide comprising a removable 3′-O-blocking group; (c) contacting the immobilized nucleotide comprising the removable 3′-O-blocking group with a deblocking agent to remove the removable 3′-O-blocking group; (d) repeating steps (b) and (c) to yield the polynucleotide; and (e) cleaving the cleavable group of the solid support to release the polynucleotide.

2. The method of embodiment 1, wherein the cleavable group is attached to the solid support via a linker.

3. The method of embodiments 1 or 2, wherein the cleavable group is a photocleavable group.

4. The method of any one of embodiments 1 to 3, wherein the solid support is a polymer chosen from polypropylene, polyethylene, cyclo-olefin polymer, or cyclo-olefin copolymer.

5. The method of any one of embodiments 1 to 4, wherein the nucleotide 5′-triphosphate comprising the removable 3′-O-blocking group has a sugar moiety chosen from ribose, 2′-deoxyribose, or 2′-4′ locked deoxyribose and a nitrogenous base chosen from a standard nucleobase, a non-standard base, a modified base, an artificial base, or an analog thereof.

6. The method of any one of embodiments 1 to 5, wherein the removable 3′-O-blocking group is chosen from (CO)R, (CO)OR, (CO)CH₂OR, (CO)NHR, (CO)CH₂NHR, (CO)SR, CH₂OR, CH₂N₃, CH₂CH═CH₂, CH₂CN, or NH₂, wherein R is alkyl or alkenyl.

7. The method of any one of embodiments 1 to 6, wherein the X family DNA polymerase is a DNA polymerase beta, a DNA polymerase lambda, a DNA polymerase mu, a DNA polymerase theta, a DNA polymerase X, a terminal deoxynucleotidyl transferase, a truncated version thereof, or a modified version thereof.

8. The method of any one of embodiments 1 to 7, wherein the deblocking agent at step (c) is an acid, a base, a nucleophile, an electrophile, a radical, a metal, a reducing agent, an oxidizing agent, an enzyme, or light.

9. The method of any one of embodiments 1 to 8, wherein the solid support comprising the free hydroxyl group and the nucleotide 5′-triphosphate comprising the removable 3′-O-blocking group are present at a weight ratio from about 1:500 to about 1:2000.

10. The method of any one of embodiments 1 to 9, wherein step (b) is performed at a temperature from about 20° C. to about 50° C. in the presence of an aqueous solution having a pH from about 7 to 9.

11. The method of any one of embodiments 1 to 10, wherein the X family DNA polymerase and unreacted nucleotide 5′-triphosphate comprising the removable 3′-O-blocking group are removed at the end of step (b) and optionally recycled.

12. The method of any one of embodiments 1 to 110, wherein the X family DNA polymerase is removed at the end of step (b) by contact with an antibody that recognizes the X family DNA polymerase.

13. The method of any one of embodiments 1 to 12, wherein step (b) is followed by a washing step to remove the X family DNA polymerase and unreacted nucleotide 5′-triphosphate comprising the removable 3′-O-blocking group.

14. The method of any one of embodiments 1 to 13, wherein step (c) is performed at a temperature from about 4° C. to about 90° C.

15. The method of any one of embodiments 1 to 14, wherein the deblocking agent is removed at the end of step (c) and optionally recycled.

16. The method of any one of embodiments 1 to 15, wherein step (c) is followed by a washing step to remove the deblocking agent.

17. The method of any one of embodiments 1 to 17, where the polynucleotide is DNA, RNA, locked nucleic acid (LNA), or a combination thereof, and has a length from about ten nucleotides to hundreds of thousands of nucleotides.

18. The method of any one of embodiments 1 to 17, wherein step (e) comprises contacting the cleavable group linked to the solid support with an acid, a base, or light.

19. A method for synthesizing a polynucleotide, wherein the method is template-independent and comprises (a) providing a nucleotide comprising a free 3′-OH group; (b) contacting the free 3′-OH group with a nucleotide 5′-triphosphate comprising a removable 3′-O-blocking group in the presence of an X family DNA polymerase and in the absence of a nucleic acid template to form an oligonucleotide comprising a removable 3′-O-blocking group, wherein the removable 3′-O-blocking group of the nucleotide 5′-triphosphate is chosen from (CO)R, (CO)OR, or (CO)CH₂OR, wherein R is alkyl or alkenyl, provided that the removable 3′-O-blocking group is other than acetyl; (c) contacting the oligonucleotide comprising the removable 3′-O-blocking group with a deblocking agent to remove the removable 3′-O-blocking group; and (d) repeating steps (b) and (c) to yield the polynucleotide.

20. The method of embodiment 19, wherein the free 3′-OH group at step (a) is at the 3′ end of an initiator sequence or an elongating polynucleotide.

21. The method of embodiment 20, wherein the initiator sequence or the elongating polynucleotide is immobilized on a solid support.

22. The method of any one of embodiments 19 to 21, wherein the nucleotide 5′-triphosphate comprising the removable 3′-O-blocking group has a sugar moiety chosen from ribose, 2′-deoxyribose, or 2′-4′ locked deoxyribose and a nitrogenous base chosen from a standard nucleobase, a non-standard base, a modified base, an artificial base, or an analog thereof.

23. The method of any one of embodiments 19 to 22, wherein the removable 3′-O-blocking group is chosen from (CO)—O-methyl, (CO)—O-ethyl, (CO)—O-n-propyl, (CO)—O-isopropyl, (CO)—O-propenyl, (CO)—O-n-butyl, (CO)—O-t-butyl, (CO)CH₂O-methyl, (CO)CH₂O-ethyl, (CO)CH₂O-n-propyl, (CO)CH₂O-isopropyl, (CO) CH₂O-n-butyl, (CO) CH₂O-t-butyl, (CO)ethyl, (CO)n-propyl, (CO)isopropyl, (CO)n-butyl, or (CO)t-butyl.

24. The method of any one of embodiments 19 to 23, wherein the X family DNA polymerase is a DNA polymerase beta, a DNA polymerase lambda, a DNA polymerase mu, a DNA polymerase theta, a DNA polymerase X, a terminal deoxynucleotidyl transferase, a truncated version thereof, or a modified version thereof.

25. The method of any one of embodiments 19 to 24, wherein the deblocking agent at step (c) is a base or an esterase or lipase enzyme.

26. The method of any one of embodiments 19 to 25, wherein the nucleotide comprising the free 3′-OH group and the nucleotide 5′-triphosphate comprising the removable 3′-O-blocking group are present at a weight ratio from about 1:500 to about 1:2000.

27. The method of any one of embodiments 19 to 26, wherein step (b) is performed at a temperature from about 20° C. to about 50° C. in the presence of an aqueous solution having a pH from about 7 to 9.

28. The method of any one of embodiments 19 to 27, wherein the X family DNA polymerase and unreacted nucleotide 5′-triphosphate comprising the removable 3′-O-blocking group are removed at the end of step (b) and optionally recycled.

29. The method of any one of embodiments 19 to 27, wherein the X family DNA polymerase is removed at the end of step (b) by contact with an antibody that recognizes the X family DNA polymerase.

30. The method of any one of embodiments 19 to 29, wherein step (b) is followed by a washing step to remove the X family DNA polymerase and unreacted nucleotide 5′-triphosphate comprising the removable 3′-O-blocking group.

31. The method of any one of embodiments 19 to 30, wherein step (c) is performed at a temperature from about 4° C. to about 90° C.

32. The method of any one of embodiments 19 to 31, wherein the deblocking agent is removed at the end of step (c) and optionally recycled.

33. The method of any one of embodiments 19 to 32, wherein step (c) is followed by a washing step to remove the deblocking agent.

34. The method of any one of embodiments 19 to 33, where the polynucleotide is DNA, RNA, locked nucleic acid (LNA), or a combination thereof, and has a length from about ten nucleotides to hundreds of thousands of nucleotides.

### DEFINITIONS

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “alkyl” as used herein describes saturated hydrocarbyl groups that contain from 1 to 30 carbon atoms. They may be linear, branched, or cyclic, may be substituted as defined below, and include methyl, ethyl, propyl, isopropyl, butyl, hexyl, heptyl, octyl, nonyl, and the like.

The term “alkenyl” as used herein describes hydrocarbyl groups which contain at least one carbon-carbon double bond and contain from 1 to 30 carbon atoms. They may be linear, branched, or cyclic, may be substituted as defined below, and include ethenyl, propenyl, isopropenyl, butenyl, isobutenyl, hexenyl, and the like.

The term “alkoxy” as used is the conjugate base of an alcohol. The alcohol may be straight chain, branched, or cyclic.

The term “alkynyl” as used herein describes hydrocarbyl groups which contain at least one carbon-carbon triple bond and contain from 1 to 30 carbon atoms. They may be linear or branched, may be substituted as defined below, and include ethynyl, propynyl, butynyl, isobutynyl, hexynyl, and the like.

The term “aryl” as used herein alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The terms “halogen” or “halo” as used herein alone or as part of another group refer to chlorine, bromine, fluorine, and iodine.

The term “heteroatom” refers to atoms other than carbon and hydrogen.

The term “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. They may be straight, branched, or cyclic. Unless otherwise indicated, these moieties preferably comprise from 1 to 20 carbon atoms.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “substituted hydrocarbyl, “substituted alkyl,” “substituted aryl,” and the like refer to said moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

Having described the invention in detail, it will be apparent that modifications and variations are possible without departing from the scope of the invention defined in the appended claims.

### EXAMPLES

The following examples illustrate certain aspects of the disclosure.

### Example 1. Incorporation of Nucleotides Comprising 3′-O-Carbamate or 3′-O-Ester Blocking Groups

A series of 3′-O-blocked deoxyribonucleotide 5′-triphosphates comprising carbamate or ester blocking groups was prepared, as indicated in the table below.

TABLE 1
3′-O-Carbamate or Ester dNTPs

| 3′-O-dNTP | Blocking Group |
|---|---|
| dNTP1 | —(CO)—O-methyl |
| dNTP2 | —(CO)-ethyl |
| dNTP3 | —(CO)-propyl |
| dNTP5 | —(CO)-methyl |
| dNTP6 | —(CO)—O-ethyl |

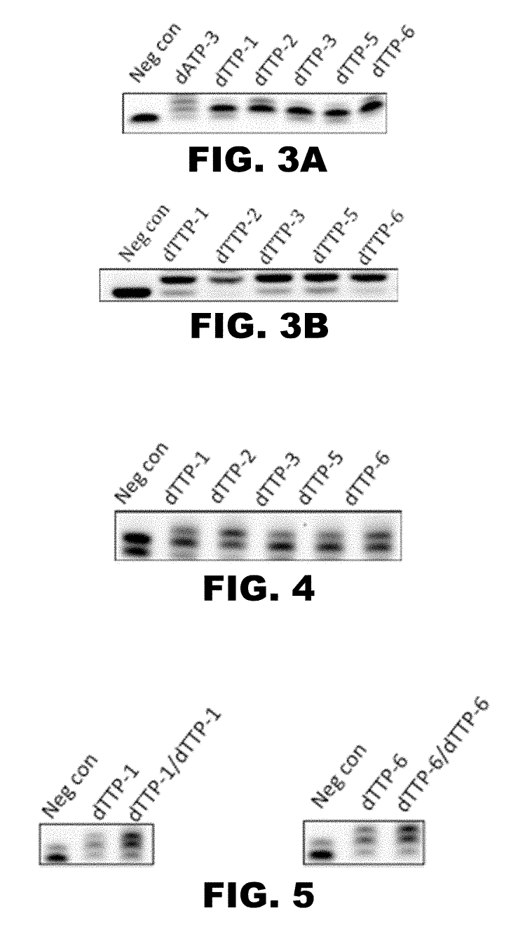

Incorporation of the 3′-O-blocked nucleotides was examined in the absence of a template sequence using Bt TdT. FIG. 3A shows incorporation of the 3′-O-blocked nucleotides into a fluorescently labeled primer in solution. FIG. 3B shows their incorporation into a similar primer that was immobilized. After the incorporate of one 3′-O-blocked nucleotide, elongation was terminated. In contrast, standard dNTPs kept being incorporated, generating oligonucleotides of varying lengths (see left lanes of FIG. 3A).

### Example 2. Efficiency of Incorporation of Different Blocking Groups

The incorporation of 3′-O-carbamate or ester blocked nucleotides was compared to that of 3′-O-azidomethyl blocked nucleotides using Bt TdT. The amount of incorporation was quantified by densitometry. As shown in Table 2, below, the carbamate or ester blocking groups exhibited at least a 6-fold increase in incorporation relative to the azidomethyl blocking group.

TABLE 2
Comparison of Blocking Group Incorporation

| | Fold increase relative to |
|---|---|
| Blocking group | azidomethyl group |
| 1 | 6.04 |
| 2 | 7.42 |
| 3 | 6.31 |
| 5 | 6.43 |
| 6 | 7.26 |

### Example 3. Incorporation of 3′O-blocked Nucleotides by Modified X Family DNA Polymerase

Human DNA polymerase mu (DNA PolM) was modified by exchanging its loop1 sequence with the loop 1 sequence of human TdT. The ability of the PolM-loop1 chimera, Hs PolM-Lp1, to incorporate 3′-O-blocked nucleotides in a template-independent manner was examined. FIG. 4 shows the incorporation of 3′-O-carbamate or ester blocked nucleotides by Hs PolM-Lp1.

The carbamate or ester blocking groups were removed by contact with heat and high pH solution (e.g., pH 12 at 70° C.). Compete removal of the blocking group was confirmed by HPLC. Multiple cycles of incorporating 3′-O-carbamate or ester blocked nucleotides using Hs PolM-Lp1 followed by deblocking are presented in FIG. 5.

### Example 4. Comparison of Mutant and Wild Type X Family DNA Polymerases

The incorporation of 3′-O-carbamate or ester blocked nucleotides by the PolM-loop1 chimera, Hs PolM-Lp1, and an N-terminal truncated PolM-loop1 chimera, Hs tPolM-Lp1, were compared to that of wild type Hs PolM. As shown in Table 3, Hs PolM-Lp1 and Hs tPolM-Lp1 showed significantly increased rates of incorporation of 3′-O-carbamate or ester blocked nucleotides as compared to wild type (WT) Hs PolM. The effect was even more dramatic with the use of a 3′-O-blocked non-natural nucleotide (d5SISC).

TABLE 3
Comparison of Mutant and Wild Type Polymerases

| | Incorporation | Incorporation | Fold increase |
|---|---|---|---|
| Blocking | Hs PolM-Lp1 | Hs tPolM-Lp1 | Hs tPolM-Lp1 |
| group | vs. WT | vs. WT | vs. Hs PolM-Lp1 |
| 1 | ++ | +++ | 2.2 |
| 2 | + | ++ | 2.1 |
| 3 | ++ | +++ | 2.0 |
| 5 | + | + | 1.3 |
| 6 (standard | + | ++ | 2.0 |
| base) | | | |
| 6 (artificial | +++++ | ++++++++++ | 3.0 |
| base - 5SICS) | | +++++++ | |

TdT does not incorporate 3′-O-blocked adenosine 5′-triphosphates very efficiently. A comparison of the incorporation of 3′-O-blocked adenosine by Hs tPolM-Lp1 and Bt TdT revealed that Hs tPolM-Lp1 exhibited a 2.7 fold increase in incorporation relative to Bt TdT.

What is claimed is:

1. A method for synthesizing a polynucleotide, wherein the method is tem plate-independent and initiator sequence-independent, and the method comprises:
(a) providing a solid support comprising a free hydroxyl group, wherein the free hydroxyl group is part of a cleavable group linked to the solid support;
(b) contacting the free hydroxyl group with a nucleotide 5′-triphosphate comprising a removable 3′-O-blocking group in the presence of an X family DNA polymerase and absence of a nucleic acid template to form an immobilized nucleotide comprising a removable 3′-O-blocking group;
(c) contacting the immobilized nucleotide comprising the removable 3′-O-blocking group with a deblocking agent to remove the removable 3′-O-blocking group;
(d) repeating steps (b) and (c) to yield the polynucleotide; and
(e) cleaving the cleavable group of the solid support to release the polynucleotide.

2. The method of claim 1, wherein the cleavable group is attached to the solid support via a linker.

3. The method of claim 1, wherein the cleavable group is a photocleavable group.

4. The method of claim 1, wherein the solid support is a polymer chosen from polypropylene, polyethylene, cyclo-olefin polymer, or cyclo-olefin copolymer.

5. The method of claim 1, wherein the nucleotide 5′-triphosphate comprising the removable 3′-O-blocking group has a sugar moiety chosen from ribose, 2′-deoxyribose, or 2′-4′ locked deoxyribose and a nitrogenous base chosen from a standard nucleobase, a non-standard base, a modified base, an artificial base, or an analog thereof.

6. The method of claim 5, wherein the removable 3′-O-blocking group is chosen from (CO)R, (CO)OR, (CO)CH₂OR, (CO)NHR, (CO)CH₂NHR, (CO)SR, CH₂OR, CH₂N₃, CH₂CH═CH₂, CH₂CN, or NH₂, wherein R is alkyl or alkenyl.

7. The method of claim 1, wherein the X family DNA polymerase is a DNA polymerase beta, a DNA polymerase lambda, a DNA polymerase mu, a DNA polymerase theta, a DNA polymerase X, a terminal deoxynucleotidyl transferase, a truncated version thereof, or a modified version thereof.

8. The method of claim 1, wherein the deblocking agent at step (c) is an acid, a base, a nucleophile, an electrophile, a radical, a metal, a reducing agent, an oxidizing agent, an enzyme, or light.

9. The method of claim 1, wherein the solid support comprising the free hydroxyl group and the nucleotide 5′-triphosphate comprising the removable 3′-O-blocking group are present at a weight ratio from about 1:500 to about 1:2000.

10. The method of claim 1, wherein step (b) is performed at a temperature from about 20° C. to about 50° C. in the presence of an aqueous solution having a pH from about 7 to 9.

11. The method of claim 1, wherein the X family DNA polymerase and unreacted nucleotide 5′-triphosphate comprising the removable 3′-O-blocking group are removed at the end of step (b) and optionally recycled.

12. The method of claim 1, wherein the X family DNA polymerase is removed at the end of step (b) by contact with an antibody that recognizes the X family DNA polymerase.

13. The method of claim 1, wherein step (b) is followed by a washing step to remove the X family DNA polymerase and unreacted nucleotide 5′-triphosphate comprising the removable 3′-O-blocking group.

14. The method of claim 1, wherein step (c) is performed at a temperature from about 4° C. to about 90° C.

15. The method of claim 1, wherein the deblocking agent is removed at the end of step (c) and optionally recycled.

16. The method of claim 1, wherein step (c) is followed by a washing step to remove the deblocking agent.

17. The method of claim 1, where the polynucleotide is DNA, RNA, locked nucleic acid (LNA), or a combination thereof, and has a length from about ten nucleotides to hundreds of thousands of nucleotides.

18. The method of claim 1, wherein step (e) comprises contacting the cleavable group linked to the solid support with an acid, a base, or light.

19. A method for synthesizing a polynucleotide, wherein the method is template-independent and comprises:
(a) providing a nucleotide comprising a free 3′-OH group;
(b) contacting the free 3′-OH group with a nucleotide 5′-triphosphate comprising a removable 3′-O-blocking group in the presence of an X family DNA polymerase and in the absence of a nucleic acid template to form an oligonucleotide comprising a removable 3′-O-blocking group, wherein the removable 3′-O-blocking group of the nucleotide 5′-triphosphate is chosen from (CO)R, (CO)OR, or (CO)CH₂OR, wherein R is alkyl or alkenyl, provided that the removable 3′-O-blocking group is other than acetyl;
(c) contacting the oligonucleotide comprising the removable 3′-O-blocking group with a deblocking agent to remove the removable 3′-O-blocking group; and
(d) repeating steps (b) and (c) to yield the polynucleotide.

20. The method of claim 19, wherein the free 3′-OH group at step (a) is at the 3′ end of an initiator sequence or an elongating polynucleotide.

21. The method of claim 20, wherein the initiator sequence or the elongating polynucleotide is immobilized on a solid support.

22. The method of claim 19, wherein the nucleotide 5′-triphosphate comprising the removable 3′-O-blocking group has a sugar moiety chosen from ribose, 2′-deoxyribose, or 2′-4′ locked deoxyribose and a nitrogenous base chosen from a standard nucleobase, a non-standard base, a modified base, an artificial base, or an analog thereof.

23. The method of claim 22, wherein the removable 3′-O-blocking group is chosen from (CO)—O-methyl, (CO)—O-ethyl, (CO)—O-n-propyl, (CO)—O-isopropyl, (CO)—O-propenyl, (CO)—O-n-butyl, (CO)—O-t-butyl, (CO)CH₂O-methyl, (CO)CH₂O-ethyl, (CO)CH₂O-n-propyl, (CO)CH₂O-isopropyl, (CO) CH₂O-n-butyl, (CO) CH₂O-t-butyl, (CO)ethyl, (CO)n-propyl, (CO)isopropyl, (CO)n-butyl, or (CO)t-butyl.

24. The method of claim 19, wherein the X family DNA polymerase is a DNA polymerase beta, a DNA polymerase lambda, a DNA polymerase mu, a DNA polymerase theta, a DNA polymerase X, a terminal deoxynucleotidyl transferase, a truncated version thereof, or a modified version thereof.

25. The method of claim 19, wherein the deblocking agent at step (c) is a base or an esterase or lipase enzyme.

26. The method of claim 19, wherein the nucleotide comprising the free 3′-OH group and the nucleotide 5′-triphosphate comprising the removable 3′-O-blocking group are present at a weight ratio from about 1:500 to about 1:2000.

27. The method of claim 19, wherein step (b) is performed at a temperature from about 20° C. to about 50° C. in the presence of an aqueous solution having a pH from about 7 to 9.

28. The method of claim 19, wherein the X family DNA polymerase and unreacted nucleotide 5′-triphosphate comprising the removable 3′-O-blocking group are removed at the end of step (b) and optionally recycled.

29. The method of claim 19, wherein the X family DNA polymerase is removed at the end of step (b) by contact with an antibody that recognizes the X family DNA polymerase.

30. The method of claim 19, wherein step (b) is followed by a washing step to remove the X family DNA polymerase and unreacted nucleotide 5′-triphosphate comprising the removable 3′-O-blocking group.

31. The method of claim 19, wherein step (c) is performed at a temperature from about 4° C. to about 90° C.

32. The method of claim 19, wherein the deblocking agent is removed at the end of step (c) and optionally recycled.

33. The method of claim 19, wherein step (c) is followed by a washing step to remove the deblocking agent.

34. The method of claim 19, where the polynucleotide is DNA, RNA, locked nucleic acid (LNA), or a combination thereof, and has a length from about ten nucleotides to hundreds of thousands of nucleotides.
